(19) United States
(12) Patent Application Publication
(10) Pub. No.: US 2026/0131164 A1
(43) Pub. Date: May 14, 2026

(54) Small-Angle Particle Beam Delivery and Positioning System

(71) Applicant: Pyramid Technical Consultants Inc., Waltham, MA (US)

(72) Inventors: Raymond Paul Boisseau, Waltham, MA (US); Andrew Dart, Swampscott, MA (US)

(21) Appl. No.: 19/389,475

(22) Filed: Nov. 14, 2025

Related U.S. Application Data
(60) Provisional application No. 63/720,400, filed on Nov. 14, 2024.

(51) Int. Cl. A61N 5/10

(52) U.S. Cl. CPC A61N 5/1069; A61N 5/1049; A61N 5/1065; A61N 5/1081; A61N 2005/1061; A61N 2005/1087; A61N 2005/1095

(57) ABSTRACT

A beam delivery system for particle therapy is provided, which when combined with an appropriate patient positioning system, can effectively replace the large and cumbersome gantry systems traditionally used in particle therapy for example for treatment of cancer or other diseased tissue conditions. The system's modular design enables scalability, making particle therapy more accessible and affordable for a broader range of healthcare facilities and patients.

### RELATED APPLICATIONS

This application claims priority to U.S. Provisional Application No. 63/720,400, filed on Nov. 14, 2024, which is hereby incorporated by reference.

### TECHNICAL FIELD

The present invention relates to a system for delivering therapeutic particle beams such as ion beams to a target in a patient where the beam source and patient position are configurable with respect to each other in an effective, compact and economical way.

### BACKGROUND

Radiation therapy (RT) uses ionizing radiation to treat malignant and other diseased tissues. In charged-particle radiation therapy, high-energy ions are directed toward a target region within a patient to deliver a prescribed therapeutic dose while minimizing exposure to surrounding or intervening healthy tissue. Accurate delivery of a three-dimensional dose distribution is important and continues to be an active area of technological development.

Particle therapy (PT), a form of external beam radiotherapy, employs charged particles such as protons, alpha particles, or heavier ions. These particles exhibit a depth-dose characteristic that enables precise placement of energy deposition within a patient. Unlike X-ray therapy, where energy is deposited continuously along the beam path, charged particles release a large fraction of their energy near the end of their range, enabling improved sparing of tissue outside the target region or target volume.

Despite its clinical advantages, particle therapy typically relies on substantial beam delivery infrastructure. Conventional systems use mechanically large gantry structures that rotate around the patient to deliver the therapeutic beam from multiple angles. These gantries are complex, heavy, and expensive, and their installation often requires specialized facilities with reinforced foundations and radiation shielding.

In a typical configuration, a high-energy charged-particle beam travels initially along a horizontal path and is redirected by a sequence of bending magnets mounted on the gantry. Additional magnetic elements are used to shape and scan the beam across the target region according to a prescribed treatment plan. A high magnetic field strength is required for the charged particle beam bending magnets to achieve a relatively small bending radius and thus reasonable overall gantry dimensions. However, there is a limit to the field strength that can be achieved practically due to the saturation characteristics of the ferromagnetic materials used to shape and direct the magnetic fields. A typical maximum field strength is around 1.6 to 1.7 T for useful magnet pole gap dimensions. Higher fields can also take more time to establish and stabilize and require larger and more expensive power supplies. Particle therapy requires the beam energy to be changed frequently during a treatment, and thus the fields of the dipoles must change correspondingly.

Referring to FIG. 1, a beam delivery arrangement 10 is illustrated in which a charged-particle beam 100 travels from left to right along an initial beam axis coincident with the gantry axis 120. The beam 100 is influence by and redirected away from the axis 120 along a beam trajectory 112. As the beam passes through successive bending magnets 110 it is directed back towards the intended target at an isocenter 140 by a series of such magnets 110 along trajectory or path 112. The isocenter 140 may be placed at a position where diseased tissue in a subject is located so as to deliver a dose of therapeutic energy to the target and cause a therapeutic effect. In the illustrated configuration, the beam 100 is transported through a sequence of magnetic elements 110 arranged along a rotational gantry structure which can rotate about its axis 120 as shown. The beam path 112 follows a trajectory established by magnetic fields generated by the bending magnets, which act to alter the momentum vector of the incoming particles while substantially maintaining beam quality.

To cause the treatment beam to enter the target from different orientations or directions, the gantry on which the system is mounted on and is rotatable about a gantry axis 120 to allow angular reorientation of the exit beam 130 relative to a stationary patient or treatment isocenter 140. By rotating the gantry, the final beam exit direction can be varied about the target, such as between a +90-degree entry position and a −90-degree entry position, represented schematically by dashed trajectory 114. The isocenter 140 defines the point in space about which the gantry rotates and at which the therapeutic beam is intended to converge during treatment delivery. For clinical purposes, current systems go through great effort and cost to allow a treatment beam to travel along conventional trajectories, which results in a substantial size and cost.

This type of beam redirection approach employs mechanical rotation of a large (and costly) structural gantry to access multiple beam entry angles. The use of sequential high-field bending magnets enables significant total beam deflection from the initial beam direction 100 to the final delivery direction 130; however, the physical size and number of magnetic elements contributes to substantial gantry mass and infrastructure requirements. In some cases the clearance to rotate such large gantries could span a distance D 150 of 10 or 20 meters or more, and without specially constructed rooms the gantry would not be able to clear the floor beneath or the ceiling above.

Furthermore, large and heavy gantries of existing systems require longer time to move about during a treatment session and even require more energy to cause said movement on account of the weight of the gantry and its gantry support systems. The larger motors needed for such movement are thus also expensive to buy, operate and maintain.

Present systems provide a horizontal patient support system (PPS) or treatment table 160 on which a patient (and the target) is placed. The table 160 is capable of rotation about a vertical axis 162 so as to increase the aspects available to treating the target. More flexible treatment aspects and beam access capabilities without undue system and gantry size and cost are desirable.

Clinical considerations including safety, stability and patient comfort have caused full gantry systems to be the preferred method of beam delivery. However, the very high expense of such systems has limited the use of PT overall. An alternative to the full gantry is the fixed beamline, in which the gantry is eliminated, and an enhanced PPS is used to perform the entire positioning task. These systems impose some limits on the types of treatments that are possible and are therefore restricted to subsidiary treatment rooms used for a more limited set of treatments or for non-clinical research work. In some examples, a PT delivery system is provided in which the patient is positioned in an upright posture allowing a CT imager to be scanned along the vertical axis. Not all treatments or patients can be placed in this configuration, and it is not universally favored.

Achieving compact gantry geometry requires strong magnetic fields to bend the therapeutic beam. However, practical limits on magnet field strength, arising from material saturation and power delivery constraints, impose minimum bending radii that contribute to overall gantry size. Frequent energy changes during treatment further complicate gantry design and operation. Existing gantry systems require large volumes and spans and weigh hundreds of tons, necessitating large treatment vaults and substantial capital investment.

The size, complexity, and cost of conventional particle therapy gantries limit the accessibility and widespread adoption of charged-particle therapy. Accordingly, there is a need for alternative beam delivery architectures that reduce system footprint and cost while maintaining clinical capability. This disclosure addresses these and other challenges in practical therapy system designs and installations.

### SUMMARY

To overcome many of the shortcomings of the prior art, the present disclosure addresses a novel particle beam delivery system and environment, which can be used for example in clinical treatments of diseased tissues in subjects. Among other aspects, the present system addresses the size, cost and related challenges arising from conventional large beam gantry and control apparatuses. Also, the interplay between beam direction and target positioning and orientation are addressed to enable the use of smaller, more cost effective and faster particle beam therapy equipment. Other advantages and features are described below.

Systems and methods are disclosed for delivering charged-particle radiation using a small-angle beam delivery system configured to operate cooperatively with a patient support or positioning system (PPS). In contrast to conventional gantries, which rely on large mechanical structures to achieve 90° or greater beam deflection around the patient, the disclosed system uses a reduced beam deflection angle between approximately 10° and 45° while preserving clinically equivalent angular treatment access. This is enabled by coordinated motion between the compact beam delivery system and the PPS, which supports controlled adjustment of patient orientation relative to the beam trajectory.

In certain embodiments, the system includes a beam transport structure to guide a charged-particle beam along a defined beam path, a rotation assembly to provide azimuthal reorientation of the beam axis, and a scanning assembly to position the beam within a target region. A control system maintains coordinated operation between the beam delivery system and the PPS to achieve non-coplanar beam trajectories suitable for conformal treatment. The disclosed approach reduces system footprint, complexity, and cost while maintaining clinical flexibility comparable to conventional gantry-based systems.

One or more examples are directed to a system for delivering a particle beam to a target, comprising a particle beam source providing a particle beam along an initial beam axis; a plurality of sequentially arranged beam deflection magnets, each beam deflection magnet in said plurality having a respective magnetic strength and orientation to cause a respective deflection of said particle beam as it passes from one such beam deflection magnet to another along a beam path that starts with said initial beam axis and ends with an exit beam axis; wherein at least a first beam deflection magnet has a corresponding first magnetic strength and first orientation to cause a first deflection of said particle beam diverging away from said initial beam axis, and at least a second beam deflection magnet has a corresponding second magnetic strength to cause a second deflection of said particle beam converging back towards said initial beam axis; wherein said exit beam axis defines an exit beam angle of less than 60 or even 45 degrees between the initial beam axis and the exit beam axis; wherein said plurality of beam deflection magnets are disposed on a gantry that is rotatable about a gantry axis that is parallel to said initial beam axis; and an articulated target support platform rotatable about at least two target support platform axes, including at least a target support platform axis running normal to gravity and normal to said exit beam axis, each such target support platform axis providing a respective degree of freedom to position and orient said target volume at a target position and target orientation crossing said exit beam axis so as to deliver a dose of energy from said particle beam to said target.

### BRIEF DESCRIPTION OF THE DRAWINGS

In the drawings:

FIG. 1 shows a conventional beam therapy apparatus according to the prior art;

FIG. 2 shows an exemplary arrangement of beam, gantry and target positioners;

FIG. 3 shows a simplified beam and deflection path about a polar axis of rotation;

FIG. 4 shows coplanar and non-coplanar beam applications;

FIG. 5 shows retractable beam nozzle and medical imager configurations;

FIG. 6 shows a rotated annular targeting and treatment zone;

FIG. 7 shows exemplary imaging and treatment examples;

FIG. 8 shows exemplary beam conditioning and positioning;

FIG. 9 shows redirecting a beam in the XZ plane using a pair of X axis deflectors;

FIG. 10 shows an exemplary four quadrupole beam conditioner; and

FIG. 11 shows exemplary beam geometries.

### DETAILED DESCRIPTION

As used herein, the term “charged-particle beam” refers to a beam of accelerated particles, such as protons, deuterons, alpha particles, or heavier ions, suitable for therapeutic radiation delivery. The term “beam transport structure” refers to magnetic and mechanical components that define and guide the beam path from a beam input to a beam output. The term “patient positioning system” (PPS) refers to a system configured to support and adjust the position and orientation of a patient relative to the therapeutic beam.

The systems disclosed herein depart from the existing systems by utilizing a reduced total magnetic beam deflection angle and achieving angular beam flexibility using programmably controllable cooperative motion between a compact beam delivery module and a patient positioning system. This approach allows clinically useful beam angle capability without requiring large gantry rotation mechanisms, without requiring massive gantry support and containment structures, and without requiring large motors to drive such large gantries as in the prior art. Several other notable innovations and features of the present system will also be illustrated and described herein.

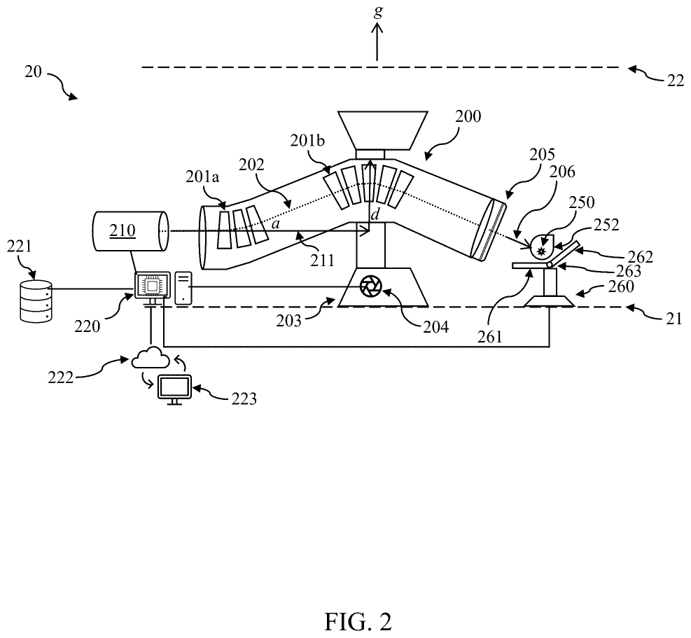

We now refer to FIG. 2, in which a charged particle beam therapy and patient positioning and target support system 20 is shown. Not every element or component of the system 20 will be necessary in every embodiment or implementation of the present invention, and in some implementations those skilled in the art will modify, adapt or augment those things described here as one skilled in the art may to achieve a specific end result without departing from the scope or spirit of this disclosure and invention. These alternate variations and embodiments are comprehended by the present invention.

The system 20 is generally disposed in a laboratory or industrial or clinical setting, treatment facility or other environment, which is typically constrained by the usual construction conditions including being set on a ground level or floor 21, and a ceiling or upper surface 22 bounding the environment's volume, which are usually horizontal with respect to the direction of gravity g. In an aspect, the present system requires much less space and clearance height (floor-to-ceiling) to install and operate compared to existing gantries used in prior art beam therapy systems. In examples of the present system, it can be fully installed and operated within conventional room dimensions, e.g., 9-foot ceiling heights. Those skilled in the art will appreciate that some or all of the examples and concepts here can be adapted for other environments and that the system can be re-designed or oriented or installed in a vertical or other configuration without departing from the nature of this disclosure. Therefore, the present illustrative examples, which mainly assume a horizontal space and placement of the main components as illustrated in the examples is not strictly limited to this situation. The drawings provided are not necessarily to scale and as such are intended for the purpose of explanation of the construction and operation of the exemplary embodiments.

The system 20 may rely on an existing particle beam source or may comprise its own particle beam generator 210 as a source of therapeutic charged particles to generate a beam of said charged particles having a given energy and flux. A suitable beam is thus delivered along an initial beam axis 211, which in the drawing travels from left to right as it enters the present system's gantry 200.

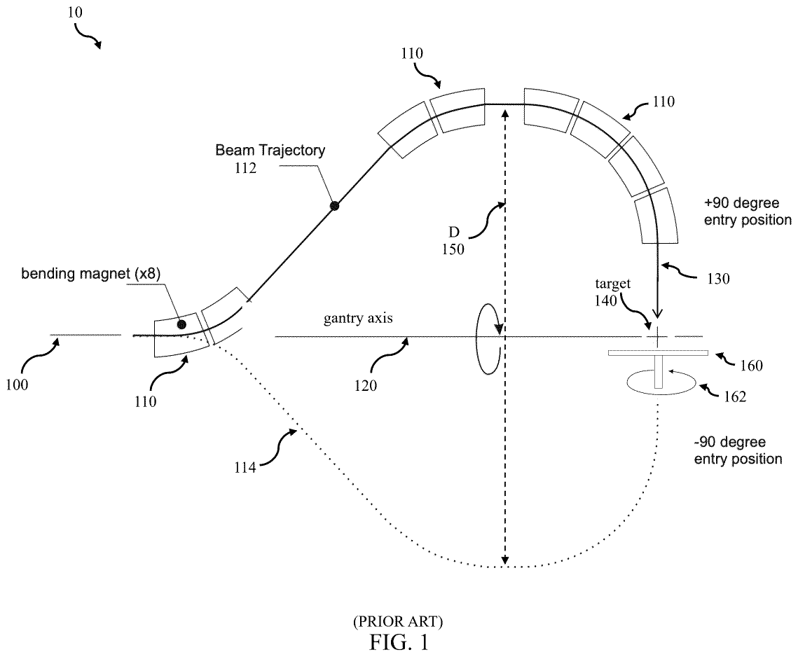

Gantry 200 itself is a moveable apparatus that comprises a frame or structure and a plurality of beam deflection magnets or beam deflectors 201. The rigid frame may be formed from a welded steel structure, a space frame assembly, or a composite structure selected to provide high stiffness-to-weight performance to limit beamline deflection during rotation and dynamic motion. The individual beam deflectors 201 are serially or sequentially arranged along the desired beam path 202 so that the beam enters into, through or proximal to the beam deflectors 201 sequentially over the course of its journey through the gantry 200. Each beam deflector 201 has a respective magnetic strength and orientation within said gantry 200 and thus makes its own contribution to the overall change in direction of the beam along the path 202. In the example shown, one or more (e.g., three) beam deflectors 201 are provided to cause an initial diverging deflection of the beam near the entrance of the gantry 200. In an aspect, the overall deflection (change in direction) of the beam from its initial beam axis 211 by a first one or more deflectors 201a, is indicated as angle a, which can be between 15 and 45 degrees. In a more specific example, the beam is deflected at an early portion of its path, by one or more deflectors 201a, by about (plus-or-minus 10 percent) 22.5 degrees. The beam reaches a maximal distance from its original beam axis 211 somewhere between the beam's entry point into the gantry system and the beam's exit point out of the gantry system. In the present invention, this maximal excursion (distance from axis 211) is much less than for conventional systems (see, FIG. 1) where the beam and gantry are required to be much larger on account of the need to fully return the beam towards the final target at an angle substantially 90 degrees with respect to the central initial beam axis 211. In the present system, the beam path 202 is turned back by a second set of one or more deflectors 201b so that it bends back or converges back towards axis 211. It is noted that the initial beam axis 211 may be the same as (coincident or congruent with) an axis of rotation of gantry 200, or substantially parallel thereto. While FIG. 2 is provided as a simplified illustration of the main components of an exemplary embodiment, it is shown at a moment in time and does not depict the rotation of gantry 200 during a treatment procedure. It will be described how gantry 200 is configured and arranged to rotate about a gantry axis elsewhere herein.

Gantry 200 is supported by and can be articulated, rotated and/or translated on a gantry support platform 203. The gantry support platform 203 is structurally robust and capable of supporting the weight of the gantry and associated parts, as well as capable of affecting any rotation of the gantry and beam delivery system during operation. One or more articulating, pivoting, sliding, or moving members coupled to the body or frame of said gantry are configured and arranged to be driven by a drive motor 204, or several electric motors, stepper motors, servo motors, lead screw drivers, linear or rotational actuators, etc. The use of a compact gantry support structure enables alternative installation configurations that are not feasible with conventional multi-hundred-ton gantry assemblies. For example, in some embodiments the system may be floor-mounted, with the base interface secured to standard or moderately reinforced flooring. In other embodiments, the system may be wall-mounted or ceiling-supported to accommodate limited floor space or specific treatment room layouts. The compact footprint of the system allows installation in facilities that would not support a conventional full gantry system, thereby expanding access to charged-particle therapy.

The movement (e.g., rotation) of the gantry 200 is in some aspects controlled by a controller and/or a manual or programmed control signal such as from a connected computer or processor 220. Those skilled in the art will understand the utility and variety of implementations of motion control of the gantry 200 using computer 220. The computer 220 may be local or remote from the rest of system 20 and may or may not be integrated therein. That is, the present example does not require the use or inclusion of a computer or processor-controlled controller therein, but this is given for the purpose of illustration of possible useful aspects of one or more embodiments. Moreover, computer 220 may be in signal and data communication with one or more data storage and/or program instruction data store 221 (e.g., to record log data, to store machine-readable program instructions, etc.). Again, the data store 221 may be physically local to the rest of the system or may be remote from said system and computer 220 as needed. In some cases, the system may be in data communication with remote machines, data sites, or computers 223 over a data network, cloud 222 or communication system suited to exchange signals, data, instructions or similar information.

The portions of system 20 responsible for carrying and deflecting and delivering the charge particle beam to its target 250 may be considered a beam transport structure.

In one embodiment, the present system includes a beam transport structure configured to receive a charged-particle beam from an accelerator beamline and redirect the beam along a defined path toward a treatment isocenter. The beam transport structure may include one or more bending magnets, steering magnets, or beam shaping elements arranged to produce a total beam deflection angle of between approximately 15 degrees and 45 degrees between a beam input axis and a beam output axis. In an embodiment the total beam deflection angle is equal to or about (within ten percent of) 22.5 degrees. The reduced deflection angle reduces the overall spatial excursion of the beam path, thereby allowing the beam delivery and mechanical support structure to be significantly smaller than that of conventional gantries that typically employ high beam deflection angles of 90 degrees. We can see how the acute deflection angles (diverging and re-converging), limit the overall lateral distance d needed to carry out the present invention, and thus result in a significantly more compact gantry and system.

Therefore, as illustrated, the particle beam travels under the influence of the one or more deflectors 201 through the body of the beam transport structure, exiting the gantry by way of a treatment nozzle 205, which is described in more detail in other publications and filings by the present inventors. After the system and beam transport structure specifically discharge the charge particle beam is travels along a final path defined by an exit beam axis 206. Target 250 is positioned in the exit beam axis path so as to receive a dose of energy from said particle beam. It is explained herein how a finite volume about target 250 can receive energy over time by moving a subject containing said target 250 and/or moving said gantry 200 to cover or paint an integrated energy deposition map in a volume we can refer to as a treatment or target volume 252, which contains the target 250 as one point therein.

System 20 further comprises a target support platform 260, which can include a base and moving members that provide one or more degrees of freedom to move and/or reorient target 250 with respect to the incident beam. This platform 260 may also be controllable manually and/or by a processor such as a computer and associated motion controller, and may be motorized as appreciated by those skilled in the art. In an aspect, the target support platform can be considered as or to comprise a patient positioning system (PPS) if a subject is to be placed and positioned thereon for treating a target or target volume within said subject. In other aspects, the target support platform 260 comprises a support table 261. In still other aspects, the table 261 may comprise two or more sub-sections, each of which can move, pivot or articulate with respect to the others. For example, an articulable portion 262 may rotate about a hinge or pivot 263 so as to place a subject in a fully or partially seated position. Various degrees of freedom are possible using the several articulating and rotating or translating members in the target support platform 260 that provide corresponding degrees of freedom for movement and positioning of target 250 and target volume 252.

Any or all of the moving parts of the gantry or target support components may be configured and arranged to yield continuous movement or stepped movement.

The beam delivery or beam transport structure may be supported by a rotation assembly and drive mechanism configured to rotate the beam output axis through an azimuthal angular range relative to the patient. The drive and rotation assemblies may comprise one or more electric motors, gears, bearings and auxiliary mechanical and electrical components to provide a torque to cause the structure to rotate about its gantry axis at a desired angular speed. In some embodiments, the rotation axis may be substantially coincident with, parallel to, or offset from the incoming particle beam input axis. The rotation assembly provides partial angular flexibility, while additional angular access is achieved by adjusting the patient position using the PPS, as described below.

In contrast to conventional gantry-based architectures that rely on large mechanical rotation about a patient to achieve a range of beam entry angles, the disclosed system utilizes a compact beam transport structure configured to provide a beam deflection angle substantially less than that of a traditional gantry, in combination with controlled patient orientation adjustment provided by a patient positioning system (PPS). Through coordinated motion between the beam delivery system and the PPS, the system is capable of achieving therapeutic beam access comparable to that of a full-rotation gantry system while significantly reducing mechanical complexity, size, and installation requirements.

The present gantry uses a total beam deflection angle much less than the conventional 90-degree gantry. By reducing the angle to a smaller value (e.g., between 15 and 45 degrees, preferably about 22.5 degrees), the overall size of the gantry structure is substantially reduced. The smaller entry angle created by this change is not a limitation of the invention on account of the present technique having a highly coordinated target support platform, e.g., a patient positioning system (PPS), having respective one or more degrees of freedom to position and orient the posture of the target subject (e.g., a patient). In some embodiments, the present system may use a vertical subject position, seated position (semi-upright) or combination of positions and orientations to suit a given application. A PPS that can change from vertical to horizontal or positions in between completes the clinical system and maintains the required features of a clinical system.

The target support platform 260 generally, or in the example of therapeutic treatment systems, the patient positioning system (PPS), supports and controls position and orientation of a target or target volume (e.g., in a patient) during operation (e.g., during a treatment session). Where the target support platform is a PPS, the target support table comprises a patient support platform. The implication here is that the present system could be used for treatment of diseased tissue (e.g., tumors) in a patient but can also be used to irradiate or dose other types of targets, living or not. As mentioned, the system can have one or more motion stages, and one or more positioning actuators configured to adjust the translational and rotational orientation of the patient or target relative to the beam output. The motion stage may provide at least three degrees of translational freedom and one or more rotational degrees of freedom (e.g., yaw, pitch, and roll). In one embodiment, the PPS is configured to rotate a patient around a patient rotation axis parallel to gravity g that is coincident with or proximate to the treatment isocenter at target 250.

In an aspect, the present system can achieve a clinically useful range of non-coplanar beam entry angles. Unlike large conventional gantries, which rely solely on rotating the beam around the patient by 90 degrees or more, the disclosed system achieves equivalent angular flexibility by dividing rotational responsibility between the beam delivery system (using the gantry platform equipment) and the target support platform (e.g., PPS). As a result, the physical size, weight, and cost of the system are significantly reduced while maintaining full clinical functionality.

In an aspect, the present system may comprise a pair of orthogonally arranged scan magnets and that generate time-varying magnetic fields to deflect the beam in a controlled pattern across the target region. The scan magnets and may be driven by a scan controller, which receives beam position commands from a treatment control system based on a prescribed treatment plan. Conceptually, these scan magnets may be in addition to or among the magnet elements (201 et seq.) depicted in the present illustrations, and the scanning controller may be implemented similar to or within the computing elements (220 et seq.) described and illustrated herein. The scanning enables precise delivery of the therapeutic dose through sequential positioning of the beam at a plurality of discrete treatment spots within a target region, passing back and forth to paint out a dose map in two or three dimensions, or for other beam control purposes.

Beam monitoring elements may be positioned in or adjacent to the scanning assembly to verify beam delivery accuracy. In some embodiments, a beam position monitor and a dose monitor are disposed along the beam path between the scan magnets and the beam output (e.g., at nozzle 205). The beam position monitor may detect the instantaneous position of the charged-particle beam, and the dose monitor may measure delivered beam current or integrated dose. Feedback signals from the monitoring elements may be transmitted to the treatment control system to support closed-loop beam delivery control. In certain embodiments, the treatment control system may dynamically adjust the scan magnet drive signals in response to detected deviations between intended and actual beam position or intensity, thereby maintaining accurate dose placement.

Computing modules local or remote to the present system (e.g., processor and machine-readable instructions at 220 et seq.) can operate and be programmed to carry out a treatment plan and may be considered a treatment planning subsystem. In some embodiments, the treatment control system communicates with a treatment planning subsystem via a communication link 222. The treatment planning subsystem 220 may be configured to generate a radiotherapy treatment plan based on patient imaging data, treatment objectives, and clinical constraints. The treatment plan may specify target volume boundaries, prescribed dose distributions, and allowable beam entry orientations. The treatment planning or control subsystem 220 may convert a treatment plan into synchronized control instructions for the scanning assembly 200, the gantry support platform, and the PPS 260 to deliver a prescribed therapeutic dose over time and space.

As described, during a particle beam treatment procedure, a prescribed beam entry orientation is achieved by a combination of partial rotation of the beam transport structure or gantry 200 and by the PPS 260. Rather than relying on a single large mechanical structure to rotate the beam through a wide angular range, the disclosed system distributes angular motion between the beam delivery system and the PPS. In an example, the gantry support platform 203 and its rotation assembly may rotate the exit beam axis 206 through a moderate angular range, while the PPS 260 rotates the target (patient) support table 261 around its vertical axis parallel to g to achieve non-coplanar beam angles relative to one another. Through this programmably controllable cooperative configuration, the system 20 provides access to a wide range of beam entry directions that would conventionally require a full 90-degree gantry deflection. In some embodiments, the treatment control system 220 may coordinate continuous or stepwise rotational motion of the beam transport structure and PPS 260 to deliver a sequence of treatment fields according to a treatment plan. The system may therefore deliver conformal dose distributions while maintaining a compact configuration and reduced mechanical complexity.

In some embodiments, the small-angle particle beam delivery system 20 is further configured to operate in conjunction with existing accelerator infrastructures without requiring modification to the accelerator itself. For example, the system may interface with a fixed horizontal beamline output from a synchrotron or cyclotron, and the reduced beam deflection architecture minimizes beamline length and magnetic complexity compared to large gantry designs. In certain implementations, the beam transport structure may be constructed as a modular assembly, enabling installation in treatment rooms with limited access dimensions. This modularity may further allow factory pre-alignment of beamline components to reduce installation time and on-site calibration requirements.

The system may optionally include a nozzle assembly 205 mounted adjacent to, at or downstream of the beam output to support beam collimation and range control. In some embodiments, the nozzle assembly 205 may include beam shaping apertures, range compensators, or energy modulation devices configured to adjust the penetration depth of the charged-particle beam according to a treatment plan.

Aspects of the invention utilizing said nozzle 205 can also incorporate some or all of the following features: An advanced pencil-beam scanning system to quickly and controllably redirect or scan the beam in one or more dimensions across a beam exit aperture, e.g., in a rectilinear, polar or other fashion. This may comprise one or more high-speed electromagnets that deflect beamlets into a series of precise spots within the patient. The nozzle may further comprise a translation or retraction mechanism to advance or withdraw the endpoint of the beamline away from its isocenter, providing clearance to allow an imaging system to move into position at isocenter. This structure utilizes a linear bearing system, flexible vacuum bellows, and a precise actuation system. The system may further employ an optional helium-filled beam path, spanning the space from the ion-beam vacuum window to the final detector system. This allows the scan magnet fields to be changed quickly without inducing eddy currents in a metal vacuum beam tube.

Overall, the present system can employ a conventional detector for providing real-time monitoring and control of the ion particle beam. These detectors may incorporate thin-film ion chambers.

The present system may also comprise a support structure or patient-specific structures such as range shifters and collimators. This may involve a motorized structure allowing software programmable configuration. This feature takes advantage of the stiffness of the primary structure to maintain accuracy.

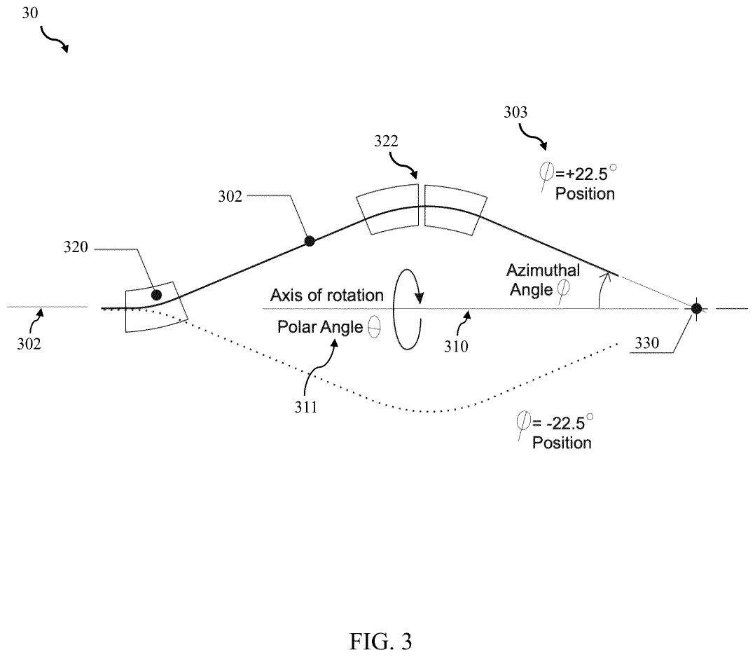

FIG. 3 illustrates a simplified view of a particle beam path or trajectory and deflection scheme according to an exemplary embodiment 30. We have an incoming beam of charged particles (e.g., ionized particles, protons, or others) 302 initially moving into the system from left to right, for example as provided by a beam generator, source or accelerator. The beam is first deflected away (diverging) from its initial incoming beam path 302 (upwards in the figure) by one or more first beam deflection or bending magnets 320, which are configured and arranged to have a beam bending power or magnetic strength to achieve the desired deflection (e.g., about 22.5 degrees) azimuthally from axis 310. The beam is azimuthally deflected away from (diverging) and back towards (converging) its initial travel axis 310 by a maximal azimuthal angle 303 (e.g,, 22.5 degrees), which can be described as +/− such maximal azimuthal angle 303. In an aspect, the present maximal azimuthal deflection angle is kept to a small amount (e.g., less than 45 degrees) compared to prior systems which incur a large deflection so as to enter the target approximately normally (at 90 degrees) with respect to the initial beam axis direction. One or more second beam bending magnets 322 achieve a second complimentary azimuthal beam deflection (converging) back towards an isocenter or target 330. The gantry and beam and beam bending magnets are rotatable about a gantry rotation axis, which may be the same as the initial beam axis 310. In some embodiments, the system can thus delivery a therapeutic particle beam to target 330 from a plurality of polar angles 311 so as to achieve a cone of energy incidence at or about the location of target 330 in what can be called a treatment volume or treatment zone, which will be described below.

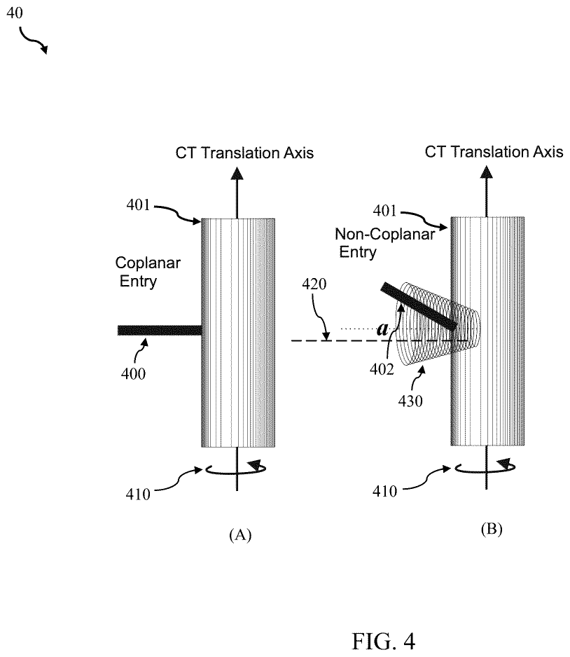

FIG. 4 compares coplanar beam entry to a target (A) and non-coplanar beam entry to a target (B). As shown in 40(A), a treatment beam 400 arrives at its target in the first (coplanar) instance in a direction normal to an axis of rotation 410 of said target. If the beam 400 delivers an energy dose to the target, and the target subject 401 is rotated about its axis 410, the region of dose delivery would appear as a horizontal disk (or narrow vertical cylinder section if the beam 400 has a finite cross section). By contrast, we see non-coplanar beam entry at 40(B) where the beam 402 is delivered by a gantry rotating about a polar axis (e.g., as shown in the preceding two figures). Here the beam 402 enters at a non-normal azimuthal angle a with respect to the axis of rotation of the gantry 420. The result in 40(B) is that we achieve a conical or conic section 430 profile of energy deposition into subject 401. This energy deposition profile or volume may be advantageous in some therapy procedures and may offer a greater treatment volume, uniformity, economy, speed and safety level compared to other treatment geometries.

The disclosed system achieves beam orientation flexibility using coordinated motion of the beam gantry and the target support platform and does not rely on large-radius rotational mechanics as prior systems did. This approach provides beam access comparable to conventional gantries while maintaining a substantially reduced system diameter. In one representative embodiment, the maximum system radius may be less than half that of a traditional 90-degree gantry, enabling installation in smaller treatment facilities or multi-room therapy centers where space constraints would otherwise prohibit the use of charged-particle therapy systems. The reduced structural complexity also lowers mechanical inertia, which may improve beam delivery responsiveness and treatment field setup time.

The systems and methods described herein provide multiple advantages compared to conventional beam delivery systems. In some embodiments, the small-angle beam delivery architecture reduces overall system mass, cost, and installation complexity by eliminating the need for large-radius gantry rotation. Additionally, the cooperative motion approach using a patient positioning system when using the system for particle beam therapy enables a wide range of beam entry orientations in a compact footprint, preserving clinical treatment flexibility. The reduced facility requirements offered by the disclosed system may increase access to charged-particle therapy by enabling deployment in standard radiation oncology facilities without extensive structural modification.

In an example, the gantry structure can be rotated about its initial beam axis, horizontal in general, and parallel to or the same as the incoming beam axis 211, allowing the azimuthal angle to vary continuously between +/−22.5 degrees from horizonal. The polar rotation angle can vary over 360 degrees but in an embodiment can rotate by about 270 degrees. This gantry polar rotation angle can be limited to 90, 180 or 270 degrees for example. For rotational angle of less that 360 degrees the specific starting and ending points can also take an any value. The small size of the present compact gantry structure allows these angle changes to be fast compared to conventional gantries with large dimensions, weight, inertia and large motor drivers. A feature of clinical interest here is the ability to move the beam over a range of entry angles during treatment to spread the dose to healthy tissue over a larger volume. This “arc therapy” is very effectively done using the system described here.

In addition to the cost reduction, this large reduction in size yields improved speed, precision, and simplicity for optimized non-coplanar radiation delivery. It can deliver a cone-shaped treatment beam profile by rotating around the patient while keeping the beam focused at a virtual isocenter within the patient. Analysis suggests that such a compact gantry system, when combined with a suitable PPS, serves the requirements of PT systems in clinical use. This assertion is based on published analyses of clinical operations. Noncoplanar entry angles are those that enter the body in a plane that is coplanar with the CT imaging planes, and so do not represent the shortest distance to the treatment point. Compared to coplanar beam arrangements, non-coplanar setups can offer significant dosimetric advantages, sparing of organs at risk (OAR).

The disclosed design has advantages over existing designs. As to cost reduction, the small-angle design results in a mechanical structure that can fit in a conventional radiation therapy room because of its small height, typically under 3 meters. This means that existing non-specialized constructions or facilities can be used in many cases. The smaller size and reduced complexity results in lower manufacturing and maintenance costs. As to speed of operation of the present gantry, the compact size and resulting small moment of inertia allows the structure to be rotated quickly and precisely. This allows the implementation of arc therapy in which the beam trajectory and the PPS are moved in synchronization during treatment, further reducing the dose to healthy tissue.

In some aspects the present system allows treatment and imaging in place. The ability to place a CT or similar imaging system at the treatment position is generally limited by mechanical interferences between the imager and the treatment structure. The disclosed design includes a moveable structure containing the final scan and monitoring nozzle. This structure can be retracted during the imaging process and then extended into place after imaging is complete. The compressible element is a highly flexible membrane bellows that is part of the vacuum system.

The present system further offers enhanced targeting precision. A unibody open-frame support gantry structure is very stiff, minimizing deflection errors and improving beam position accuracy. The addition of heavy post-scan beamline accessories is simplified. The structure includes the ability to retract the final mechanical structure and to restore its original position with high accuracy. This allows the critical imaging and alignment system to share the same location as the patient treatment, providing the capability of imaging the patient in the exact treatment position. This is a highly desired feature that enhances accuracy in the clinical setting. Dynamic adjustments to beam intensity and angle enable superior dose distribution with high efficiency, potentially reducing radiation exposure to surrounding tissues.

The present design can be fully counterbalanced in rotation, eliminating the need for active braking systems, simplifying the mechanical drive system and improving safety. The heavy counterweight used here is structured to contain the rack of sensitive electronics used in the real-time delivery of the ion beams in clinical use. The structure offers effective radiation shielding while allowing sensitive electronics to be positioned close to the detectors. The retractable structure is designed to enhance safety for the patient by preventing potential collisions between the patient and the nozzle.

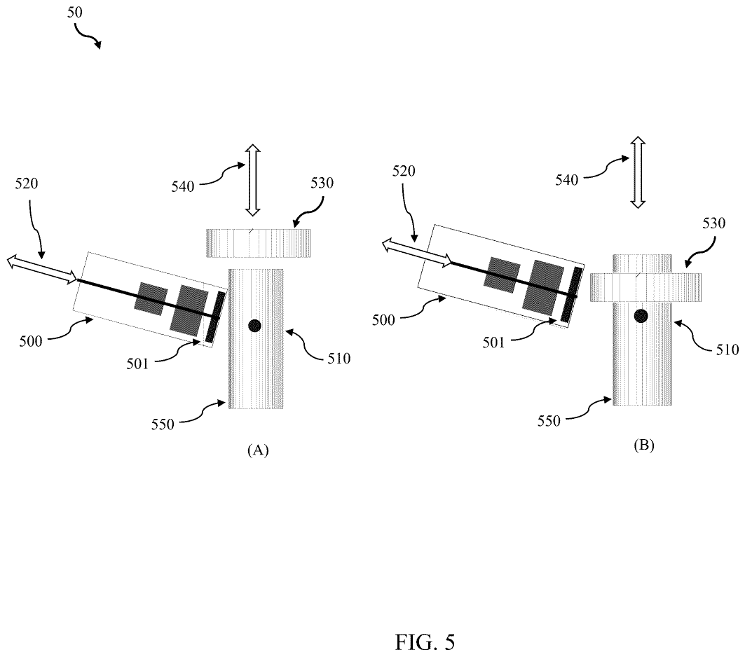

FIG. 5 illustrates a portion of the present system 50 according to some aspects where the beam delivery system 500 terminates in a nozzle assembly 501 before exiting the delivery system along an exit path to meet the target at the isocenter 510. The nozzle assembly 501 may be retractable (along an axis 520) to increase clearance around the treatment isocenter at target 510 during patient setup or imaging procedures. In such embodiments, the nozzle 501 may be moved into a deployed position (A) during treatment and withdrawn to a retracted position (B) during imaging workflows, allowing flexible integration with imaging systems such as cone-beam CT, MRI, or X-ray imaging devices 530. Likewise, imaging system 530 may be translated in a complimentary way along an imager translation axis 540 so that it is out of the way of treatment nozzle 501 during delivery of a treatment beam (A) and is moved towards or around subject 550 during active imaging (B). That is, nozzle 501 or proximal parts of the particle delivery system 500 may be moveable in some embodiments to permit advancing or retracting the exit portion of the beam delivery system towards or away from subject 550 and/or isocenter target 510. In the case where patient or subject imaging is to be accomplished in conjunction with a treatment, such imaging apparatus (e.g., a CT scan head) 530 may thus have adequate physical clearance in the vicinity of the subject/target to acquire images thereof without interference from the beam delivery 500 or nozzle 501 system.

In an embodiment, the present system delivers a therapeutic particle beam to a patient isocenter, target or target volume at an angle being about 22.5 degrees to the initial beam axis or gantry platform rotation axis. The converging or latter or descending leg following the maximum radial divergence of the beam can be long enough to contain the treatment nozzle, which includes an X, Y (face of exit plane) beam scan system and monitoring detectors. An air gap between the end of the nozzle and patient isocenter is a compromise between air scatter and clearance and is typically about 80 cm during treatment. These are merely exemplary figures of course and not limiting of the embodiments of the present system.

Once the angle and descending length are determined, the remaining consideration is the minimum radius of curvature achievable by the electromagnetic dipoles. This is typically about 5 meters but is dependent on the maximum beam energy and the magnet technology. With reasonable values of these constraints, a 22.5-degree entry angle can be achieved. A first magnetic dipole deflects the beam from horizontal by 22.5 degrees. At the apex of the structure, a second magnetic dipole deflects the beam toward isocenter at an angle of 45 degrees. This yields an entry angle referred to its original horizontal trajectory of 22.5 degrees. The structure can then be rotated about the original beam axis by at least 180 degrees so that the beam trajectory now describes a cone of 22.5-degree half-angle with its apex at the patient position isocenter. The entry into the patient relative to the horizontal can now be set to +22.5, zero, or −22.5 degrees or any intermediate angle, when combined with a coordinated rotation of the PPS. The precision and stiffness of the structure is such that the intersection of the beam with the nominal horizontal axis remains accurate to under 1 mm without additional compensation.

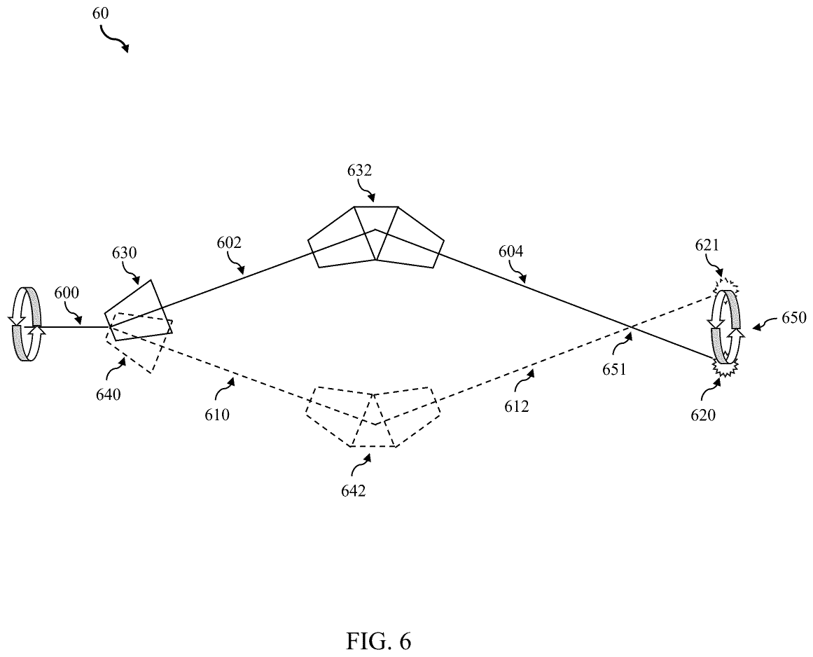

FIG. 6 shows a representation 60 of a charged particle beam path in a gantry as described herein having an overall small angle deflection (being less than 45 degrees, and preferably between 15 and 30 degrees, and preferably about 22.5 degrees) from the direction of the incoming beam 600. The solid lines and deflectors indicate the position and beam path at a first time, while the dashed lines and deflectors indicate the position and beam path at a second time where the beam gantry is rotated between the first and second time by a polar angle (e.g., 180 degrees or other amount).

At the first time, beam 600 enters a first one or more deflector magnets 630, which deflect the beam along 602 divergingly away from initial axis of beam 600 towards a second one or more deflector magnets 632. The second one or more deflector magnets 632 are at the apex or maximal radial distance of the beam at said first time and re-direct the beam convergingly back towards the isocenter 651 along 604. Note that we are assuming a very fast moving beam so that the entirety of the solid line at the first time is assumed to be substantially a snapshot. Those skilled in the art will understand that some finite, very minute, time is required for a particle in a beam to actually traverse across the gantry and the figure. The exiting beam traveling along 604 crosses a number of physical positions along this path, including geometric isocenter 651 and another position 620.

At the second time shown as dashed elements, beam 600 is deflected by deflectors 640 (which are a time rotated version of 630) so that the beam travels divergingly out along 610. Then, as before but rotated around the gantry axis, the second deflector magnets 642 (which are a time rotated version of 632) bring the beam back in convergingly along 612, through isocenter 651 and on to another position 621.

Note that if a continuous (or discretely stepped) rotation of the gantry and beam is achieved between the first and second times shown, a corresponding circular ring 650 is painted out by the particle beam, which defines an extended ring or cone shape in or at a desired target volume. In other words, the treatment location(s) of the present system do not need to be limited to an isocenter location. Rather, they can be extended through an isocenter 651, which in some embodiments permits a smaller overall system to treat a subject as the beam angle (gantry) is rotated about the gantry axis.

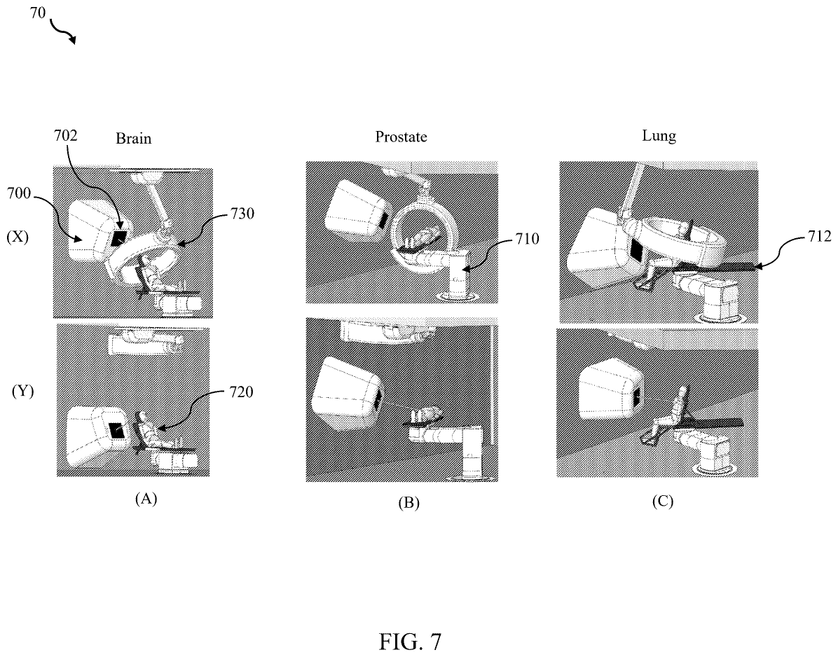

FIG. 7 illustrates a number of exemplary configurations 70 usable to couple the movement of a particle beam delivery apparatus and gantry 700 and associated patient positioner or target support platform 710, and medical imaging apparatus 730, in an integrated or coordinated system for applying an energy dose to a target volume in a subject 720, e.g., to treat a diseased organ or tissue. The present examples are provided for illustration of the many applications and configurations that may be generalized or customized by those skilled in the art in addressing specific implementations of the invention. Each of the exemplary scenarios depicted concerns the use of a controllable treatment beam and gantry 700 (showing a portion thereof, e.g., the beam delivery nozzle 702) and a medical imaging apparatus 730, which is also controllably moveable with respect to the patient subject and the other elements of the system. The upper row of frames (X) shows the patient undergoing an imaging phase of a treatment procedure, in which practitioners may get updated images of the treatment region within the patient, or automated measurements may be taken to control the progress of or termination of a treatment session. The lower row of frames (Y) shows the medical imaging apparatus retracted out of the way and the beam delivery apparatus actively delivering a therapeutic dose to a region of interest.

Scenario (A) depicts an exemplary brain therapy treatment and imaging set where (A, X) shows an imaging phase of the procedure and (A, Y) shows the imaging apparatus retracted (up) to clear the way for a proper particle beam therapy phase to treat a zone of the subject's brain, e.g., a brain tumor. Note that these phases of the procedure are not strictly exclusive. In other words, in some embodiments, imaging may take place during application of the therapeutic beam, and vice versa.

Scenario (B) depicts an exemplary prostate treatment and imaging set where (B, X) shows an imaging phase without beam application and (B, Y) shows a beam application phase without imaging.

Similarly, (C) depicts an exemplary lung treatment and imaging set where (C, X) shows an imaging phase and (C, Y) shows a beam treatment phase.

It is noted that the target support platform or patient support and positioning apparatus 710, sometimes referred to as a PPS, is actively engaged and controlled to position the subject properly for each phase of the procedure, and including actively during a beam treatment delivery phase if a physically extended or distributed treatment volume within the subject is desired. A computer-controlled motion/positioning system can translate, rotate, elevate, tilt and otherwise provide motorized orientation and positioning of the subject and target zone in concert with the corresponding movements of the beam delivery apparatus and gantry. Specifically, in one or more embodiments, the PPS 710 may comprise a target support platform such as a patient support bed 712. The platform 712 may be a one-piece table and remain in a flat horizontal configuration, such as in scenario (B) for the illustrated prostate treatment. But in other embodiments it may have one or more subsections, for example two parts that can be pivotably articulated to help a patient be raised to a seated position or inclined or partially inclined position, as in the illustrated scenarios (A) and (C) for brain or lung treatments, though these are only illustrative and not required in all cases. In this and other examples of the present system and its operation, the invention thus contemplates some treatments to employ such patient movement to place some or all of the body of a subject in a fully or partially elevated configuration (with respect to gravity) above the nominal horizontal plane. To elaborate on this point, the present system employs the polar rotation of a compact beam gantry as described to introduce a dose of energy in a particle beam incident at relatively small deflection angles from the axis of the gantry (e.g., less than 45 degrees, between 15 and 30 degrees, or about 22.5 degrees). The coordinated combined rotation of the gantry a700 and beam in conjunction with corresponding matched movements of the patient support platform apparatus 710 can access and cover a large useful variety of dose geometries in a clinical system 70. This enables out-of-plane beam incidence onto a patient compared to and in addition to conventional in-plane beam incidence delivery where the beam was made to travel in an extended trajectory to enter the patient at a 90-degree normal direction thereto. Preferred embodiments of the PPS 710 enable rotation about one or more axes, degrees of freedom, and including pivoting all or some of the subject out of the horizontal plane parallel to the ground. In combination, the geometric and dosing results needed can be achieved as described herein with a compact efficient overall system.

The optics of the beams described herein may benefit from additional magnetic optics, typically including magnetic quadrupole lens elements and small-angle deflectors to correct small trajectory variations. Variable beam energies can use either a variable energy accelerator, or a post accelerator energy selector. This function can be integrated into the present gantry by designing the ion optics to create an energy-dispersive beam waist within the structure. This may be coupled with an energy selection slit, and an energy degrader structure.

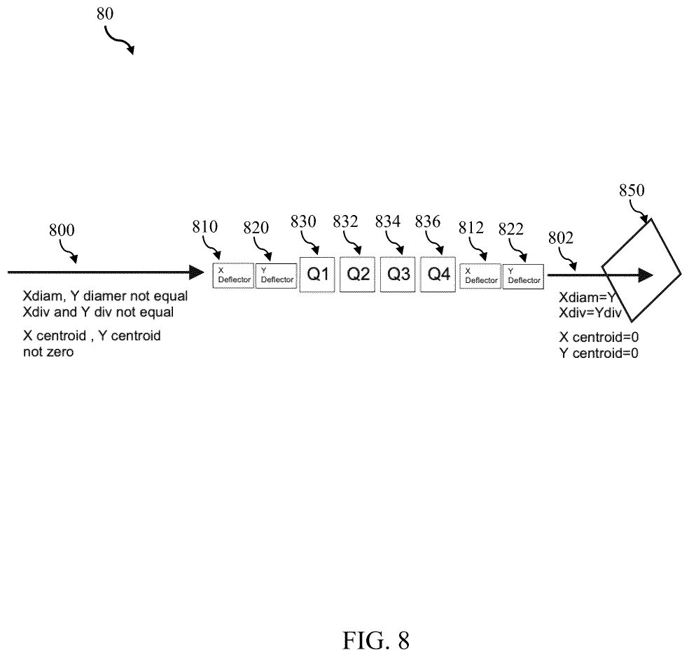

FIG. 8 shows an exemplary arrangement 80 of beam conditioning components that may be used in one or more embodiments. The initial beam path 800 is conditioned by passing through a first set of X 810 and Y 820 deflectors; followed by four quad singlets 830, 832, 834, 836, arranged in series; followed by another set of X 812 and Y 822 deflectors. The exiting beam path 802 is shown with respect to a normal plane 850 in the X-Y plane.

In an aspect, to vary the beam energy for fixed-energy ion sources, one may employ an energy degrader made up of a variable thickness of material. This may be coupled with an energy selection system made up of a magnetic energy spectrometer and defining slit. In some large systems, the energy selection system may be situated at the exit of the particle accelerator. A disadvantage of this configuration is that beams with energy spread produced by such a system cannot be transported long distances without loss. The present system may thus employ an energy selection system close to its point of use.

In one or more specific aspects, the present system conditions an incoming charged particle beam to be compatible and effective with the present beam gantry and other parts of the system. The system design comprehends that an incoming beam from the beam source may have some finite extent (diameter) in a vertical axis and a horizontal axis in the plane normal to the beam's direction of travel. Also, there could be divergence of the beam in each of these axial directions that should be accommodated. In a respect, the present system conditions the particle beam so that it is rotationally invariant, e.g., relative to an initial beam axis and/or beam gantry rotational axis. If made rotationally symmetric about its initial axis and/or the gantry's axis, the gantry can be rotated to deliver the therapeutic dose as described without alteration of the relevant parameters of the beam and allowing the optics of the system to remain unmodified during gantry rotation in a treatment session. Specifically, in an aspect, two magnetic deflectors are disposed in the path of the incoming beam in a first axis (X) of the plane normal to the beam (e.g., vertical), and another two magnetic deflectors disposed in said path in a second axis (Y) of the plane normal to the beam (e.g., horizontal) such that the first (X) and second (Y) resultant beam displacements place the beam on a desired beam trajectory straight down the axis of rotation of the system's beam gantry. In other words, a minimum of four beam deflectors (two in X and two in Y) are used to displace the beam to point in an initial beam direction or axis along the axis of rotation of the beam gantry.

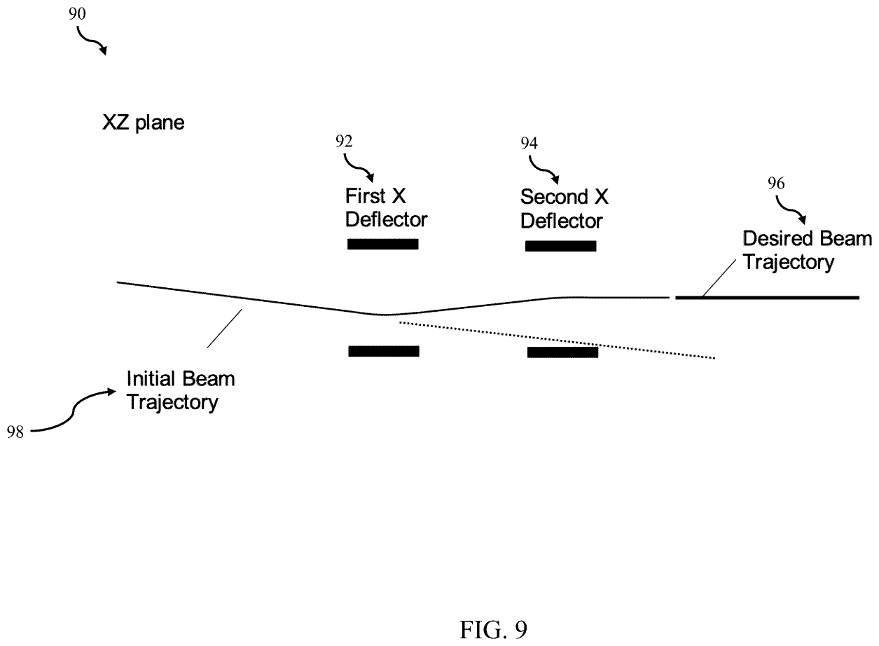

FIG. 9 illustrates the use of a pair of deflectors 92, 94, consistent with the arrangement shown in the previous figure, to redirect the beam in the XZ plane 90 so it is redirected from its beam source trajectory 98 to be along a desired beam direction 96 in said plane. A similar redirection is done in the YZ plane to redirect the beam in a desired Y direction. The end result is a centering of the beam and directing of the beam along the gantry axis. The axis of the desired beam trajectory 96 in the figure is sometimes referred to herein as an initial beam axis, i.e., at the start or prior to entering the main beam diverging deflectors of the beam gantry discussed herein. Any order of operations or steps to redirect the beam are possible, e.g., two deflections in X followed by two deflections in Y, or one in X followed by one in Y then another in X and another in Y, and so on.

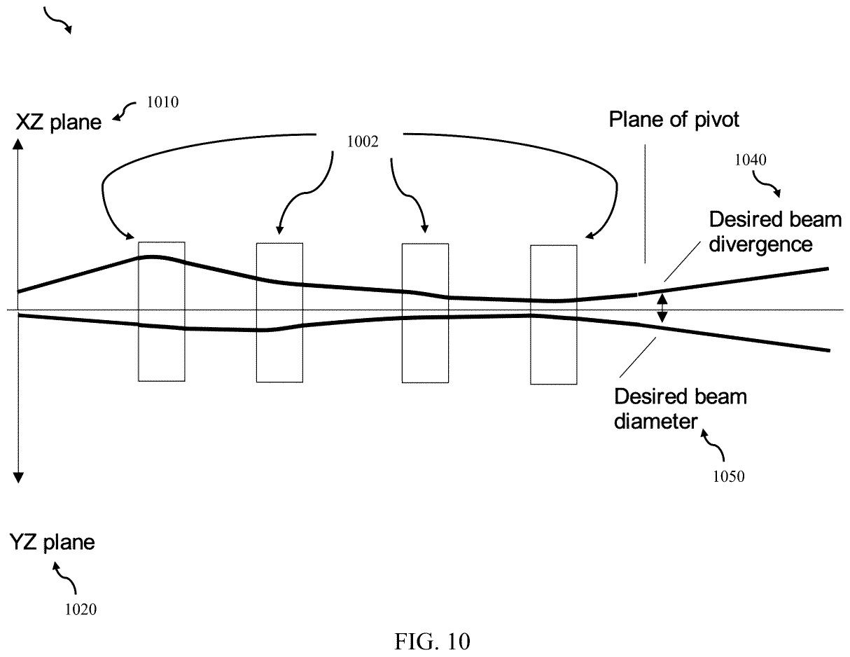

FIG. 10 illustrates a portion of a beam conditioner 1000 comprising four quadrupole magnetic lenses 1002. The upper portion of the figure depicts behavior in a first plane (e.g., vertical XZ 1010) normal to the direction of beam travel, and the lower portion of the drawing depicts behavior in a second plane (e.g., horizontal YZ 1020) also normal to the direction of beam travel. A desired beam divergence is thus achieved at 1040, and a desired beam diameter is thus achieved at 1050. Placement and direction of the beam is previously addressed. Generally, one controlling element can be used for each constraint of the beam to be achieved or modified. So in an example, changing four beam constraints may be done using four quadrupole magnetic lenses. More specifically, in this example, the four quadrupole magnetic lenses can be used to modify the diameter (along X and along Y) and divergence (in XZ and in YZ) of the beam in each of the two axes normal to the beam path, i.e., four parameters. Matching the parameters in the two axes orthogonal to the beam path allows the beam to be rotationally symmetric about the beam path for the reasons stated before.

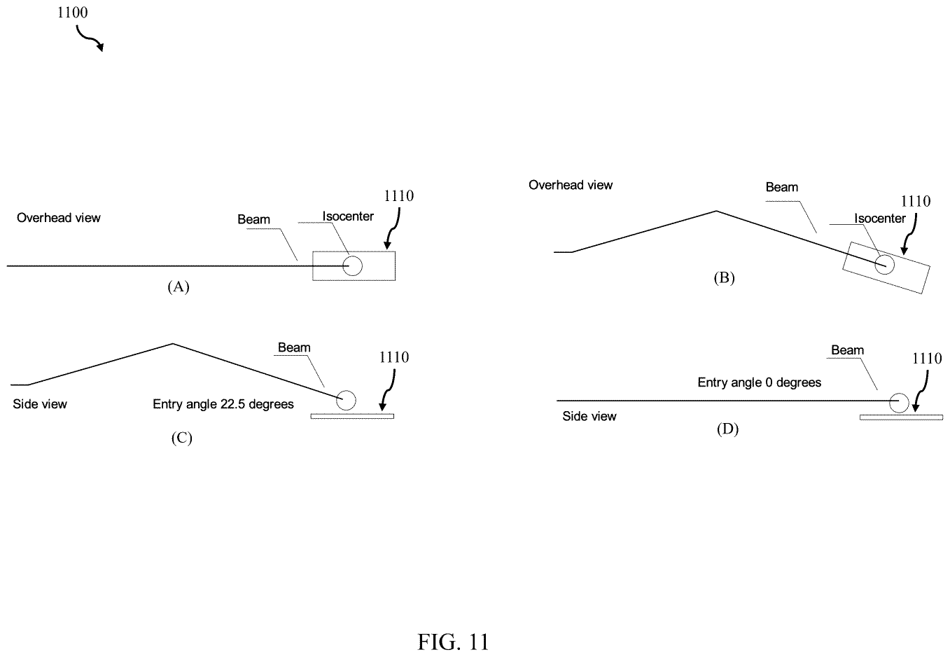

FIG. 11 illustrates several exemplary views 1100 of a beam moving through a beam gantry according to the present disclosure. Views (A) and (B) show the beam (traveling left to right) from above (an overhead view) while views (C) and (D) show the same beam from the side. The views also show the beam as it appears at two times, for example where views (A) and (C) are snapshots at a first moment in time where the body of the beam gantry and the beam is deflected in a vertical plane with respect to the ground plane while views (B) and (D) show the situation at a second moment in time when the beam gantry has been rotated by 90 degrees about its axis of rotation and at which time the body of the gantry and the divergence of the beam is in a horizontal plane parallel to the plane of the ground. The simple exemplary views assume a gantry design that deflects the particle beam by about 22.5 degrees from its initial entry axis and/or the gantry's rotation axis. Note that to maintain a treatment direction or aspect, the patient support platform 1110 rotates the patient by an equivalent angle as the gantry is rotated so as to keep a same patient to beam perspective and beam approach to the isocenter. The rotation of the patient platform can be seen from overhead, e.g., top views (A) and (B). In side views (C) and (D) we do not see this rotation in the simple drawings as the plane of the platform 1110 remains in the horizontal plane parallel to the ground.

Some embodiments may include an energy degrader at or near the entry point of the present gantry apparatus. This can be implemented as a series of insertable plates (binary degrader) or as a continuous structure such as a linear wedge or dual-wedge system. The control system for the positioning the degrader, plus the settings of the energy dispersive system (above) would allow the user to select an energy on demand, and the energy selector components would be automatically set.

It is to be appreciated that these and other specific illustrative examples are not provided by way of limitation, but rather to explain the design and operation of the present system. Those skilled in the art will understand how and where to make equivalent systems, modifications, additions or deletions to suit a specific need.

What is claimed is:

1. A system for delivering a particle beam to a target, comprising:
a particle beam source providing a particle beam along an initial beam axis;
a plurality of sequentially arranged beam deflection magnets, each beam deflection magnet in said plurality having a respective magnetic strength and orientation to cause a respective deflection of said particle beam as it passes from one such beam deflection magnet to another along a beam path that starts with said initial beam axis and ends with an exit beam axis;
wherein at least a first beam deflection magnet has a corresponding first magnetic strength and first orientation to cause a first deflection of said particle beam diverging away from said initial beam axis, and at least a second beam deflection magnet has a corresponding second magnetic strength to cause a second deflection of said particle beam converging back towards said initial beam axis;
wherein said exit beam axis defines an exit beam angle of less than 60 degrees between the initial beam axis and the exit beam axis;
wherein said plurality of beam deflection magnets are disposed on a gantry that is rotatable about a gantry axis that is parallel to said initial beam axis; and
an articulated target support platform rotatable about at least two target support platform axes, including at least a target support platform axis running normal to gravity and normal to said exit beam axis, each such target support platform axis providing a respective degree of freedom to position and orient said target volume at a target position and target orientation crossing said exit beam axis so as to deliver a dose of energy from said particle beam to said target.

2. The system of claim 1, wherein said gantry axis is congruent with and lies along said initial beam axis.

3. The system of claim 1, wherein said articulable target support platform supports a plurality of degrees of freedom to position and orient the target with respect to the particle beam, including a first degree of freedom about a first vertical support platform articulation axis parallel to gravity (yaw); a second degree of freedom about a second platform articulation axis being horizontal and normal to gravity (roll); and a third degree of freedom about a third platform articulation axis being horizontal and normal to gravity as well as normal to said second platform articulation axis (pitch).

4. The system of claim 1, said support platform comprising at least two vertical axes of rotation, a first vertical axis of rotation running through a weight bearing member supporting said support platform above ground, and a second vertical axis of rotation, horizontally offset from said first vertical axis of rotation by a radial distance therefrom.

5. The system of claim 1, said target support platform comprising a support table capable of supporting a human subject when said target is located within said subject.

6. The system of claim 5, wherein said support table comprises at least two sections that are pivotably articulable with respect to one another so that the support table can be laid horizontally flat when the at least two sections are extended in-line with each other, or pivotably articulated with respect to one another to elevate one of the two sections with respect to the other in a hinged manner.

7. The system of claim 1, wherein said exit beam angle has a value in a range between 15 and 45 degrees.

8. The system of claim 7, wherein said exit beam angle has a value of 22.5 degrees.

9. The system of claim 1, wherein said gantry is configured and arranged to be able to move through its full range of rotation about said gantry axis without interference from either of a floor or a ceiling of a room in which said system is placed.

10. The system of claim 9, wherein said gantry is configured and arranged to be able to rotate about said gantry axis by at least 180 degrees.

11. The system of claim 10, wherein said gantry is configured and arranged to be able to rotate up to a range of 270 degrees about said gantry axis.

12. The system of claim 10, wherein said gantry axis is horizontal with respect to a ground plane and said gantry is supported on a gantry support platform having a vertical extent, and wherein said gantry support platform comprises a C-shaped support structure allowing said rotation.

13. The system of claim 1, further comprising a retractable beam nozzle from which said particle beam is emitted along said exit beam axis, wherein said beam nozzle is configured and arranged to translatably extend towards said target when delivering particle beam treatment and to retract away from said target when not delivering particle beam treatment.

14. The system of claim 1, further comprising a moveable imaging head that can be moved towards the target when performing target volume imaging and moved away from the target when not performing target volume imaging.

15. The system of claim 1, wherein said gantry and said target support platform are configured and arranged to move with respect to one another so as to deliver said dose of energy to said target from a plurality of directions over a period of time.

16. The system of claim 1, wherein said gantry and said support platform are configured and arranged to move with respect to one another so as to deliver a spatially distributed dose of energy to a target volume about said target over a period of time.

17. The system of claim 1, wherein said beam source comprises a plurality of beam setup deflectors that direct and orient said beam along said initial beam axis prior to arriving at said plurality of beam deflector magnets.

18. The system of claim 1, wherein said particle beam comprises an ion beam and said beam source comprises an ion beam source.
